(19) United States
(12) Patent Application Publication
(10) Pub. No.: US 2007/0167355 A1
(43) Pub. Date: Jul. 19, 2007

(54) Dietary and pharmaceutical compositions for management and treatment of oxidative stress

(76) Inventors: Rita R. Ellithorpe, Tustin, CA (US); Vladimir I. Slesarev, Coeur d'Alene, ID (US); Todor V. Dimitrov, Chestnut Hill, MA (US)

Correspondence Address: Vladimir Slesarev, 18406 Broadleaf road, Boyds, MD, 20841, US

(21) Appl. No.: 11/581,623

(22) Filed: Oct. 17, 2006

Related U.S. Application Data
(63) Continuation-in-part of application No. 10/455,123, filed on Jun. 6, 2003, now abandoned.
(63) Continuation-in-part of application No. 10/794,285, filed on Mar. 8, 2004, now abandoned.

(51) Int. Cl. A61K 38/14

(52) U.S. Cl. 514/8

(57) ABSTRACT

Inhibition of TNF alpha prooxidant action is achieved after administration of biodegradable N-Acetyl-glucosamine-N-acetyl-muramyl-peptides released after specific endopeptidase and lysozyme digestion of the genus Lactobacillus and Bifidum. This invention also provides a medical food for dietary management of all conditions with elevated Gamma Glutamyl Transferase activity and concurrent alterations of NF-κB expression, which may be particularly useful for apoptosis modulation in people with chronic viral infection and cancer metastasis.

This patent application is a continuation in part of original application Ser. No. 10455123, filed on May 6, 2003, CIP filed on Dec. 11, 2003, and CIP Ser. No. 10/794,285 filed on Mar. 8, 2004.

### FIELD OF INVENTION

The present invention relates to apoptosis stimulating glucosamine-muramyl-peptides, obtained by specific endopeptidase digestion of gram positive bacteria, medical food compositions and methods of application thereof in dietary management, prevention, and treatment of the chronic viral infection and cancer metastasis.

### BACKGROUND OF THE INVENTION

Oxidation, that is, raising the level of the free radicals: reactive oxygen species (ROS)- the hydroxyl groups, and hydrogen peroxide is indisputably the final and universal stage of the pathogenesis of chronic degenerative diseases, and their lethal complications. These toxic radicals inflict the direct tissue damage and lead to a lethal outcome. Increase in their concentration is a result of and is proportional to the reduction in the levels of the major antioxidant glutathione, which is destroyed (through hydrolysis) by gamma glutamyl transferase GGT (transpeptidase). Its high activity level proved to be a main risk factor of cardiovascular mortality (Ruttman E., Brant L J, Concin H, et al. Circulation. 2005, Oct. 4, 112, 2130-37))

The recent studies have documented the involvement of oxidative stress in the stimulation of apoptosis. In fact, many treatments known to induce apoptosis are also to induce the formation of oxidant mediators, such as reactive oxygen species and nitric oxide. It also well established that many inhibitors of apoptosis can exert antioxidant effect action, either directly or by enhancing cellular antioxidant systems.

In parallel, the involvement of oxidant reaction in the cellular balance between apoptosis and survival appears to be more complex. In fact, evidence has been forwarded that in some cases the exposure of cell to low, non toxic levels of the reactive oxygen species, super oxide and hydrogen peroxide can exert a stimulatory effect on their proliferation, rather than promoting apoptosis or cell necrosis. Also, it has been reported that pretreatment of cells with a mild oxidative stress can result in their protection against apoptogenic stimuli (Del Bello et al., FASEB, 1999, V.13, p.2669-2678).

It has been in fact documented that gamma glutamyl transferase (GGT) activity can give rise to redox reaction, leading to the production of reactive oxygen species and lipid peroxidation (Dominici et al., 1999, Free.Rad.Biol.Biol.Med., p.623-635). In particular, the low levels of hydrogen peroxide originating as a by- product during GGT activity are capable to prevent apoptosis and maintain proliferation of histiocytic lymphoma cells (Del Bello, et al., 1999). The same mechanism was implicated in the development of liver fibrosis and diabetes during hepatitis C viral infection.

Expression of GGT has been regarded as a maker of neoplastic progression in several experimental models, such as rodent skin and liver chemical carcinogenesis. Significant levels of GGT have been reported in number of human malignant neoplasms, e,g. ovary (Paolocci, et al., 1996), colon (Murata et al., 1997), lung (Blair et al., 1997), liver (Tsutsumi et al. 1996), melanomas (Melezinek et al, 1998), leukomias (Tager et al., 1995); in many instances, GGTP levels detectable in metastasis are higher than in corresponding primitive tumors. In particular, in different clones of Me665/2 melanoma cells the degree of GGTP expression was found to correlate with the metastasis potential, as estimated from invasiveness and migration experiments in vitro (Maellaro et al., 2000, J. Cell Sci., 113, 2671-2678). The observation that comparable H2O2 amounts can originate from GGT activity of different tumor cell line, supports thus the possibility that such prooxidant function of GGT activity may represent a general feature of this enzyme. In this perspective, it is conceivable that GGT-mediated prooxidant reactions could be a general feature of those malignant neoplasms in which high levels of GGT activity have been documented to occur. GGT-mediated extracellular H₂O₂ production might participate in the endothelial damage, which is regarded as a necessary step in the establishment of metastasis (Bittinger et al., 1998). The recent studies have documented that a higher constitutive activation of NF-κB is a result of constitutive prooxidant status induced in these cells by their high GGT activity. This interpretation was strengthened by the observation that stimulation or inhibition of GGT activity in 2/60 cells indeed resulted in stimulation or inhibition of nuclear translocation of P65, respectively. Activation of NF-κB could play a major role in determining the reported higher malignancy of 2/60 cells as compared to their GGT-poor counterparts (Maellaro et al., 2000, J. Cell Sci., 113, 2671-2678). Several studies point in fact to the particular relevance of NF-κB activation in the malignant behavior of cancer cells, due to its involvement in the expression of several gene products participating in cancer invasion. Indeed, antisense inhibition of NF-κB has been shown to inhibit tumorogenecity in nude mice injected with tumor-derived cell lines (Higgins et al., 1993). On the other hand, inhibition of NF-κB by IκBα can enhance TNF alpha-induced apoptosis in prostate cancer cells, as well as confer sensitivity to apoptosis of human glioma cells (Muenchen et al., Clin Cancer Res, 2000, 6, 1969-1977, Otsuka et al., Cancer Res, 1999, 59, 4446-4452).

In addition, long-lasting sustained activation of NF-κB has been observed in chronic disorders such as diabetes type 1 and 2 and its complications (Bierhaus et al, Diabetes, 2001, 50, pp.2792-2808, Romeo et al., 2002, v.51;2241-2248). A persistent NF-κB activation has also been suggested in atherosclerosis, Parkinson and Crohn diseases, Listeria monocytogenes infection, inflammatory bowel disease (Loncar et al., Gut, 2003, 52: 1297-1303, Soos J. et al., Neuroreport, 2004, 6; 15, 1715-8).

A significant correlation between the levels of GGT and the frequency of stroke in the patients with high blood pressure was observed in epidemiological observations under the EuroStroke project (Bots M L, et al., J Epidemiol Community Health. 2002 Feb;56 Suppl 1;25-29)

Just simple presence of GGT protein was implicated as an indispensable factor for osteoclast forming activity (Niida et al., JBC, 2004, 279, 5752-5756). Furthermore, both native GGTP and inactive GGTP stimulates the expression of the receptor activator of NF-kappaB ligand MRNA and protein from bone marrow stromal cells. (Suda et al., Endocr.Rev.20, 345-357). Increased osteoclast activity is responsible for progressive bone loss in post-menopausal osteoporosis and Paget's disease (Rodman, G D, Endoc. Rev., 1996, V.17,308-332). Local bone distruction has also been observed in bone metastasis and rheumatoid arthritis (Guise T and Mundy G R, Endocr. Rev., 19, 18-54). Tumor cells that have metastasized to bone induce osteoclastogenesis via the secretion of bone resorbing factors such as PTH-related protein, IL-11, and prostaglandin E2 (Guise T and Mundy G R, Endocr. Rev., 19, 18-54).

TNF alpha, interferon gamma, interleukine 6 and interleukine 1beta are produced in the bone marrow or other organs of patients with anemia of chronic disease (ACD). It is associated with cancer, rheumatoid arthritis, multiple myeloma, non-Hodgkin lymphoma, myelodysplastic syndromes, idiopatic myelofibrosis, and end-stage. renal disease (Stenvinkel P. Nephrol.Dial.Transplant., 2001, 16: 36-40) this disease. These cytokines have been implicated in the pathogenesis of ACD because they inhibit erythropoesis while fostering the development and function of marrow cells involved in inflammation. A final common pathway for the inhibition is likely to be the induction of erythroid cell apoptosis.

Clinical evidence for the relation between TNF alpha and ACD comes from studies of monoclonal antibodies to TNF alpha, which ameliorate signs and symptoms in chronic inflammatory disease. Rheumatoid arthritis patients, who were treated with monoclonal antibodies to TNF alpha showed improvement in their anemia that was not mediated through changes in erythropoietin. (Papadaki H., et al., Blood, 2002, 100: 474-482).

In parallel, TNF alpha plays a critical role in the control of neutrophil survival by inducing an apoptotic death program which can be rapidly triggered by a variety of stimuli. When neutrophils were pretreated with TNF alpha and then were exposed to different inflammatory agents, there was a marked stimulation of apoptosis. A broad panel of stimuli which includes cytokines IFN gamma and GM-CSF was found to make a difference in triggering apoptosis of neutrophils treated with TNF alpha. By contrast, a slight increase in the number of apoptotic cells was also found, when neutrophils were cultured only with TNF alpha (Salamone G., et. al, J.Immunol., 2001, 166:3476-3483) Dysplasia of megakaryocytic, granulocytic, and erythroid lineages are the hallmarks of myelodysplastic syndromes. The apoptosis prevails kinetically over increased proliferation, causing the peripheral cytopenia. In MDS many studies link overexpression of TNF alpha to cell death (Head D R., et al., Leukemia, 1996, 10:1826, Lancet J E., et al., Hematol/Oncol.Clin.N.Am., 2000, 14:251-267). TNF alpha produced by MDS mononuclear cells is inhibitory to both normal and MDS colony growth indicating that residual normal hematopoiesis can also be blocked in MDS (Head D R., et al., Leukemia, 1996, 10:1826). The identification of TNF alpha as a key cytokine in cell death regulation and increased susceptibility of MDS cells to TNF alpha is the basis for several clinical trials of TNF-alpha inhibitors (Bennett J M, et al., Br. J. Haematol., 1982, 51:189-199).

However, one of most successful of them, recombinant TNF alpha receptor (Embrel) simply reduces level of serum TNF alpha. It proved to be a risk factor of developing sepsis and often the patients with rheumatoid arthritis are to be placed on antibiotics.

Thus, there is a great deal of need to develop medical food with antioxidant properties without affecting the beneficial role of cytokines.

### BRIEF DESCRIPTION OF THE INVENTION

The present invention is based on the discovery that biodegradable cell wall fragments of gram positive bacteria of the genus Lactobacilli and Bifidum inhibit NF-κB and gamma glutamyl transpeptidase, thus reducing oxidative stress. It increases sensitivity to both FAS ligand and TNF alpha mediated apoptosis of cancer cells. In addition, apoptosis of cell infected with RNA virus is also enhanced by newly discovered phenomena of inhibition TNF alpha stimulatory effect over NF-κB. Such reduction is selective, and does not lead to the enhanced apoptosis of the innocent bystander cells. Moreover, it reduces apoptosis of these cells via blocking proapoptotic TNF alpha action. On the contrary, the same mechanism of the blocking TNF alpha action leads to the inhibition of cellular oxidative stress, thus enhancing apoptosis both cancer cells and cells infected by virus.

Consequently, in one aspect the invention provides biodegradable glucosamine muramyl peptides in compositions with bioflavonoids and food antioxidants, which demonstrate the modulation of TNF alpha mediated oxidative stress. This modulation of TNF alpha killing pathways proved to be clinically effective in the management of all conditions with elevated GGTP levels. Concurrent inhibition of nuclear factor be of TNF alpha is useful in preventing and treatment cancer metastasis, leukemia, sepsis, diabetes, Cohn disease, atherosclerosis, and inflammatory bowel disease. Applicants also propose peptidoglycans compositions of Lactobacillus and Bifidum, which enhance the red blood cells, white blood cell, and platelets count in patients with aplastic anemia.

Another aspect of the present invention is to provide a novel medical food consisting of the probiotic peptidoglycans containing N-acetyl-glucosamine-N-Acetyl-muramyl-dipeptides-tripeptides, -tetrapeptides, -pentapeptides, -hexapeptides, -octapeptides, and bioflavonoids.

These food ingredients posses synergistic effect on the inhibition of TNF alpha cytotoxicity. On the other hand, the proposed compositions are effective in ameliorating the oxidative stress, promoting apoptosis of the cancer cells as well as the cells infected with virus. Such medical food may be used to reduce cancer and hepatitis associated leukopenia and pancytopenia. Specifically, the present food may be recommended for those patients who suffer from common postchemotherapy toxicity such as leukocytopenia, thrombocytopenia, elevated free iron, bilirubin, and liver enzymes. Further, the present invention provides nutrition for dietary management of leucopenia, cancer cachexia, muscle dystrophia , and myeilodysplastic syndrome.

In a related aspect, the present invention provides a food useful for treating patients suffering from chronic hepatitis C. Fortified food and drink may be especially beneficial. for people with concurrent liver cirrhosis, thus preventing severe fatigue and brain damage caused by ammonia. Furthermore, presented invention provides the food for metabolic detoxifications of the carcinogenic chemicals and mutagens, which lead to anemia.

Another aspect of the present invention includes nutritional methods for the management of anemia in patients with rheumatoid arthritis. Therapeutic effect is based on newly discovered phenomena of the inhibitory effects of probiotic peptidoglycans over T-lymphocytes mediated cytotoxicity. This inhibition does not lead to reducing TNF alpha level in the blood, thus eliminating the risk of septic complications and cancer. Moreover, such effects are beneficial for rheumatoid arthritis because smoothers cytotoxic TNF alpha effects, which play a crucial role in the pathogenesis of this disease.

Still another aspect of the present invention includes dietary methods of treating anemia and diabetes caused by ribovarin and interferons in the patients with hepatitis C.

While another aspect of this invention is to provide a method to treat or prevent anemia, thrombocytopenia, or neutropenia by administering to a subject having or at risk of developing anemia, thrombocytopenia, or neutropenia a combination a dietary peptidoglycans of L. Plantarum as medical food and anemia, thrombocytopenia, or neutropenia medicament. In certain embodiments, glucosamine muramyl peptides are derived from both Lactobacillus and Bifidum bacteria and food antioxidants dietary supplements are selected from the group consisting of sea urchin, papaya corica, garlic, n-acetyl-glucosamine, black and blue berries, vitamin B12, vitamin B6, vitamin C, folate, vitamin D, calcitriol, alphacalcidol, androgen, selenium, and carnitine. In a preferred embodiment glucopeptides of B.infantis are released after glycine endopeptidase hydrolysis and papaya corica is substituted with papaya Pubescences. In certain embodiments, the preferred antioxidant food is freeze dried with all ingredients lyophilized and evenly mixed.

Yet another aspect of this invention comprises oral administration of the probiotic peptidoglycans with papaya latex proteases, lysozyme, and sea urchin in order to improve bioavailability of the urchin proteins.

Still another aspect of this invention includes the application of the probiotic glucopeptides to inhibit the bone resorption syndrome. GMDP and GMTP are reducing the expression of both GGTP and NF-kappaB factor, which play significant role in pathogenesis hypercalcemia. The examples of this syndrome are the bone metastasis of prostate and breast cancer, myoloma, lymphomas, osteoporosis caused by estrogen shortage and/or rheumatoid arthritis.

Amount per serving of predigested probiotic culture in the range of from 200 mg to 4000 mg may be found to be acceptable for dietary management of the conditions with elevated GGTP with optimal range of 1500-2500 mg per day. Daily isolated petidoglycan dosage in the range of from 5 mg to 300 mg would acceptable with optimal range 30-90 mg. Still, another aspect of this invention covers newly discovered phenomena of the inhibitory effects of soy isoflavones over TNF alpha cytotoxicity. A retained natural level in the range 50-70 mg of isoflavones may be found suitable for such dietary management. A daily dose of 50-75 g of isolated soy proteins with at least 0.1 weight percent of the retained isoflavones is preferable serving quantity.

### BRIEF DESCRIPTION OF THE DRAWINGS

For further details, reference is made to the discussion which follows, in light of the accompanying drawings, wherein:

FIG. 1 illustrates the inhibition of lactate dehydrogenase (LDH) release by peptidoglycans of L. Plantarum.

FIG. 2 demonstrates the synergistic effect of soy isoflavones and peptidoglycans of L. Plantarum on LDH release.

FIG. 3 illustrates the inhibition of LDH release by N-acetyl-glucosamine-N-acetyl-muramyl-L-Alanine-D-glutamic acid (GMDPA)

### DETAILED DESCRIPTION OF THE INVENTION

The present invention relates to the dietary and pharmaceutical inhibition of TNF alpha prooxidant action, and food and drinks, containing, as an effective component biodegradable N-Acetyl-glucosamine-N-acetyl-muramyl-peptides released after specific endopeptidase and lysozyme digestion of the genus Lactobacillus and Bifidum This invention also provides a medical food for dietary management of all condition with elevated GGTP levels and concurrent alterations of NF-κB expression, which may be administered orally to humans and domestically useful animals in single dose as small as 20 mg/kg. The dosage of 100 mg/kg may be preferable. Inhibitory effects over GGTP may be enhanced by isoflavones and bioflavonoids, which are retained in isolated soy protein and dealcoholized red wine. For the safety reasons, peptidoglycans from B.infantis may be preferable.

Disaccharide tetrapeptide is the part of the basic unit of the peptidoglycans of Gram negative bacteria and L.Plantaaum. The peptidoglycan is a single bag shaped highly cross-linked macromolecule that surrounds the bacterial cell membrane and provides rigidity. It consists of glycan (polysaccharide) backbone consisting of N-acetyl muramic acid and N-acetyl glucosamine with peptide side chains containing D- and L-amino acids and diaminopimelic acid. In the cell wall they are bound to teicholic acid and polysaccharide by a phosphate diester band. Basic unit was purified by Takase et. Al., (U.S. Pat. No. 4,545,932 1985). However, under certain preparation conditions, two aminosugars (N-acetyl-glucosamine) are linked to muramic acid. This bond remains basically intact after lysozyme hydrolysis.

A great deal of endotoxicity is caused by peptidoglycans derived from gram-positive bacteria. Its peptidoglycan is able to induce leukopenia and thrombocytopenia (Verhoef J. and Kalter E., Prog.Clin.Biol.Res. 1985; 189:101-113). Moreover, peptidoglycans and lipoteichoic acid can cause the induction of nitric oxide (NO) formation, shock, and organ failure in the rat (Kengatharan K, et al. J. Exp. Med., 1998;20:305-15). Disaccharide tetrapeptide, N-acetyl-glucosamine-muramyl-L-alanine-D-isoglutamine-meso-diaminopimelyl-L-alanine was shown to be cytotoxic in explanted hamster tracheal tissue and hamster tracheal epithelial cell culture (Luker K E., et al., Proc.Natl.Acad.Sci., 1993, 90: 2365-2369). This disaccharide tetrapeptide induces leukocytosis in cerebrospinal fluid, influx of protein into CSF, or brain edema, alone or in combination. Muropeptide carrying the diaminopimelyl-diamninopimelic acid cross-link specifically induced cytotoxic brain edema (Burroughs M, et al., J.Clin.Invest., 1993, 92:297-302). The structural analog N-acetyl-glucosaminyl-1,6-anhydro-N-acetylmuramyl-L-alanine, disaccharide dipeptide (GMTP), is responsible for synergizing with lipoteicholic acid thereby causing septic shock during gramm positive infection. Orally administered peptidoglycans enhance leukopenia by stimulating of the phagocytosis of splenetic neutrophils from mice. (Sasaki T., et al. J.Vet.Med.Sci. 1996, 58:85-6). In addition, peptidoglycans, well known tumor necrosis factor (TNF) alpha stimulators, promote rheumatoid arthritis inflammation (Simelyte E., et al., Infection and Immunity, 2000, 68:3535-3540).

It is well known fact that low molecular weight peptidoglycans, GMTP and GMDP (695 D), are mainly responsible for immunogenic effects. They are weak stimulators of TNF alpha production, which may be useful prevention of septic shock, but at the same time could be detrimental for the patients with autoimmune conditions such as rheumatoid arthritis. Excessive level TNF alpha production could be harmful patients with ARDS, stroke, and ischemic heart disease, who already have high preexisting production of TNF alpha Moreover, combination of MDP and TNF alpha can cause proinflammatory effects, thus exaggerating chronic viral and bacterial infection.

The present invention has been completed on the basis of findings that stimulation of apoptosis is based on inhibition of GGT and NF-κB expression in cancer cells by biodegradable N-acetyl-glucosamine-N-acetyl-muramyl-peptides without concurrent inhibition of TNF alpha production.

In parallel, apoptosis of innocent bystander cells is inhibited via suppression of TNF alpha cytotoxic action.

Biodegradable glucosamine-muramyl- peptides of the general formula N-acetyl-glucosamine-N-acetyl-muramyl-L-Ala-D-isoGlu-R1-R2 R3 or N-acetyl-glucosamine-N-acetyl-muramyl-L-Ala-D-isoGlu-R1-, where R1 is lysine or ornithine residue, R2 is amino acid residue selected from the group of D-alamine, L-alanine, D-aspartic acid, L-glycine, L-serine, D-serine, and L-threonine, R3 is amino acid residue selected from the L-glycine, D-asparagine, D-glutamine are found to be inhibitors of GGT and NF-κB of the cancer cells.

Their antioxidant effect is caused by the presence of two D-aminoacids, D-isoglutamine and D-alanine. They can be prepared after lysozyme and specific endopeptidase hydrolysis. Preferable are N-acetyl-glucosamine-n-acetyl-muramyl-L-Ala-D-isoGlu-(Glu)-L-Lays-D-Glu-D-Ala Gly and N-acetyl-glucosamine-N-acetyl-muramyl-L-Ala-D-isoGlu-L-Lays-D-Ala. They released after digestion of probiotic culture of B. infantis with lysozyme and glycine endopeptidase.,

Most preferable is N-acetyl-glucosamine-n-acetyl-niuramyl-L-Ala-D-isoGlu (Glutamic acid) [GMDP (GMDPA)], released after digestion of all Lactobacilus and Bifidurm bacteria with lysozyme and endopeptidases V8 and Lyt F.

These biodegradable peptidoglycans modulate functional activity of the natural killer cells via inhibition GGTP expression on their membranes. It can lead to reduction in the numbers of these T lymphocytes, when their population is dangerously increased. This effect could be beneficial for those autoimmune conditions, where T killers are significantly elevated and cause tissue damage.

In parallel, undesirable proinflammatory and immunogenic properties were avoided by adding soy isoflavones, red wine, and cocoa bioflavonoids. They can act synergistically with glycopeptides. Thus, such composition enhances the inhibition of TNF alpha cytotoxicity and reduces adverse side effects of TNF alpha stimulation. It provides exceptional safety and improved tolerance in people with autoimmune conditions. In addition, achieved GGTP inhibition leads to the repletion of glutathione, well-known TNF alpha inhibitor.

The present invention provides the oral inhibitors of GGT and TNF-α induced oxidation, thus desensitize malignant cell to apoptosis. In parallel, they inhibit NF-kappaB expression, which along with the presence of GGTP plays significant role in the development of bone resorption syndrome.

The glycopeptides fraction of molecular weight of not higher than 3,000 D and not less than 300 D in the cell wall of gram positive bacteria may be purified by a known method of molecular weight fractionation of proteins or by ultrafiltration. The presented peptidoglycans for GGT inhibition with concurrent inhibition of immunogenicity are significantly different from previous inventions related to probiotic peptidoglycans. All of them have presented probiotic peptidoglycans as the immunostimulators. Immunostimulatory properties were reported by Link and Pahud (U.S. Pat. No. 5,185,321, 1993) and by Yamazaki et al, (EP99104209, 1999). Moreover, Yamazaki et al. taught to increase immunogenicity by purifying low molecular weight fraction of 500-4000 peptidoglycans with increased percentage of very low molecular weight of 500 D peptidoglycans. Their main objective was to increase production of TNF alpha to the level comparable with the stimulation by muramyl dipeptide (MDP). More over, the structure of the peptidoglycans is different: there is lysine instead of diaminopimelic acid.

The apoptosis modulating compositions and anemia, neutropenia, or thrombocytopenia medicament can be administered in a synergistic amount effective to treat or to prevent anemia, thrombocytopenia, or neutropenia.

In some embodiments of the invention, the peptidoglycans are-administered to in the effective amount to treat or prevent aplastic anemia caused by chronic viral infection. In this aspect of the invention, the GMTP containing peptidoglycans are administered to the subject to restore destroyed hematopoieses, caused by accelerated apoptosis. Anemia, neutropenia, or thrombocytopenia medicament is subsequently administered to the subject. This method is particularly useful in subjects who are particularly susceptible to bacterial or viral disease, such as children, immunocompromised subjects, and elderly subjects.

In other aspects, the method of the invention involves administering a high dose of an anemia, thrombocytopenia, or neutropenia medicament to a subject, without inducing side effects. Ordinarily, when an anemia, thrombocytopenia, or neutropenia medicament is administered in high doses, a variety of side effects can occur. As a result of these side effects, the anemia, thrombocytopenia, or neutropenia medicament is not administered in such high doses, no matter what therapeutic benefits are derived. It was discovered, according to the invention, that such high, doses of anemia, thrombocytopenia, or neutropenia medicaments which ordinarily induce side effects can be administered without inducing the side effects as long as the subject also receives a peptidoglycan.

The peptidoglycan modulators of TNF alpha of the present invention have the following excellent features;
1. They are inhibitors of TNF alpha cytotoxicity originated from. lactic acid bacteria and Bifidum, which are used in the production of yogurt and fermented milk food and drinks.
2. They are the substances from natural origin which can sensitize cancer cells to apoptosis to a dose much less than the known TNF alpha inhibitors produced by plants, sea weeds and microorganisms.
3. Since they are water soluble, they can be readily prepared in appropriate formulations. Latest feature is a real benefit for parenteral administration.
4. In composition with-predigested urchin protein their effects over stimulated by TNF alpha oxidative stress can be enhanced. Such food compositions also provide well-balanced daily source of the amino acids.
5. They are stimulators of apoptosis of the cells infected with RNA viruses.
6. They detoxify opiates, anesthetics, and alcohol.
7. They are inhibitors-of bone resorption syndrome by reducing GGTP and NF-κB expression.

As stated above, GGT inhibition leads to the preservation of extracellular glutathione -powerful antioxidant with remarkable detoxification properties. In particular, the invention has application in alcohol detoxification, anesthetic recovery and in recovery or withdrawal from hypnotics, narcotics, sedatives or other drugs, especially in case of abuse. Treatment of withdrawal is a particular area where the invention has applicability.

The invention may have application in the prevention, treatment or management of toxicity caused directly or indirectly by one of the following compounds:
anesthetics, including: local anesthetics (such as cocaine, procaine, lidocaine, tetracaine, mepivacaine, bupivacaine and etidocaine, chlorprocaine), inhalational anesthetics (such as methoxyflurane, halothane, enflurane, isoflurane and nitrous oxide); intravenous anesthetics (etomidate, benzodiazepines, and barbiturates);
opiates, including: heroin and morphine related opiates (such as hydromorphone, oxymorphone, levorphanol, codein, hydrocodon, oxycodone, nalorphine, naloxone, naltrexone, buprenorphine, butorphanol and nalbuphine);
sedatives and hypnotics, including barbiturates and benzodiazepines;
other drugs subjects to abuse, including cocaine and related drugs; nicotine and tobacco; —psychedelic drugs, which are hallucinogenics and/or psychotomimetics and/or psychotogenics;
ethanol and its metabolites.

The invention has applications in dealing with endogenous created toxins. Acetaldehyde, the primary metabolite of ethanol, is an example. Probiotic glycoprotein is useful for dietary management of the patients who accumulated endotoxins as a result of disease.

One endogenous toxin, which can often cause the problems, is bilirubin. High levels are known to results in jaundice, particularly in babies and patients with advanced hepatic metastases. Reduced liver function is also a characteristic feature of geriatric patients. In addition, in cancer patients, levels of drug such as analgetics and chemotherapeutics tend to build up in the body and this can lead to severe side effects. Yet, other endotoxins, free radicals, are generated during radiation and chemotherapy.

However, the applicants have been able to demonstrate that the level of metabolites such as bilirubin and iron in the blood of patients could be significantly reduced when patients are fed with probiotic glycoprotein.

Because of their exceptional inhibitory activity over GGT and NF-κB, biodegradable peptidoglycans are valuable food for combating the highly oxidative tumors with significant metastasis potential. Most preferable applications are hepatocellular carcinoma, ovarian carcinoma, colorectal cancer, and glioblastoma The proposed food compositions would be also beneficial for ameliorating flee radical toxicity in patients who already have extensive metastasis growth to the liver or brain. In parallel, leukocytopenia and thrombocytopenia, which are common toxic side effects of radiation and chemotherapy in cancer patients, can be corrected. Precisely how leukocytopenia and thrombocytopenia are treated and prevented remains to be shown. Though, it is well established fact that the inhibitors of TNF alpha cytotoxicity, such as anti TNF alpha antibodies, an agent used to treat ulceratus colitis, prevents myelosuppresion in cancer patients treated with melphalan (Gupta V., et al. Cancer Chemother. Pharmacol. 1995; 36:13-9).

From the above it can be seen that the invention also relates to a method for the prevention, treatment and management of the chemoresistant GGT positive tumors with liver, brain, and bone morrow damage mediated by TNF alpha In addition, the invention relates to nutrition for reducing damage after ribovarine treatment of hepatitis C.

Feeding with probiotic peptidoglycans is particularly effective in those patients who have increased risk of aplastic anemia caused by chronic viral infection. Patients with severe thrombocytopenia after chemotherapy also can benefit from proposed medical food by improving both platelets and neutrophil count. It can be beneficial for patients with septic shock, where anemia and leucopenia are life threatening complications.

Yet, among other indications is anemia caused by autoimmune diseases such as rheumatoid arthritis. The present inventor has carried out intensive research in order to develop safer agents, which can exhibit profound desensitizing effect without significant immunosupression. Consequently, he has found out that the decreasing percentage of low molecular weight peptidoglycans by ultrafiltration of ingredients less than 10000 D and higher than 30000 D creates glycoprotein composition which clinically does not lead to the symptoms of overproduction of TNF alpha, thereby is absolutely safe for people with rheumatoid arthritis. The present invention provides a medical food, which can desensitize T lymphocytes without any immunosuppression. The method for preparing medical food according to the present invention will now be explained. The GMTP compositions to be used in the present invention may be obtained from a variety of gram positive bacteria of genus Lactobacillus or Bifidum,. Following own methods. The amino acid sequence and sugar composition of the complexes will be defined by a species of bacterium.

Lactobacillus is cultured via the culture conditions suitable for the microbiological properties of the species, to collect the cultured bacterial cells. These may be cultured in the culture medium routinely used for lactobacillus, for example, Rogosa medium, but complex medium using soy protein broth or distiller's soluble, etc. as nitrogen source may be also used. Peptones, yeast extracts, and glucose are most preferable ingredients of culture medium. The fermentation methods may follow the routine methods for lactobacilli. Routine methods for bacteria degradation such as ultrasound, temperature (hot water), and enzyme hydrolysis may be employed. Lysozyme hydrolysis is preferable one.

Routine methods for purifications of peptidoglycan complexes can be employed. More specifically, hydrolysate obtained by aforementioned methods, is applied to anion-exchange column to remove lysozyme and high-molecular nuclear acids. Further, protease and nuclease can be used for degradation of the remaining proteins and nuclear acids, respectively. Hydrophobic chromatography may be used to remove enzymes by passing them through a column with resin. Glycoprotein composition may be fractioned by gel chromatography.

Yet, nanofiltration by using 100 D, 200, or 300 D membranes and ultrafiltration by using membrane with cut off range of 3000 D-500000 D is considered most preferable method for purifications of the peptidoglycan compositions. More specifically, 50000 D polyethersulfone membrane may be used to filtrate the nuclear acids and high molecular weight proteins from aforementioned lysozyme hydrolysate. Then, 1000 D membrane may be employed to eliminate salts and water from the composition. This high molecular fraction is indicated for anemia treatment in patients with autoimmune diseases, when immunogenic, proinflammatory peptidoglycans with low molecular weight may cause severe side effects.

Similarly, fractions, which compose of different percentage of low molecular weight glycopeptides, can be obtained by using 100 D reverse osmosis membrane and 3000 D or 10000 D polyethersulfone membranes. 1000 D membranes are used for filtrating low molecular pyrogenic muramyl peptides and glucomuramyl peptides, respectively, as well as salts and acetic acid. Employing 10000 D or 30000 D membranes can regulate percentage of high molecular weight peptidoglycans. Fractions obtained by aforementioned ultrafiltration are especially effective for inhibition of TNF alpha cytotoxicity in patients with anemia caused by chronic viral infection.

The bacterial wall preparations can be obtained by routing method for their separation with ion detergents. A fraction, which contains up 98% of any particular glucosamine muramyl peptides can be purified by preparative HPLC.

The preferred peptidoglycan compositions can be obtained by mixing with sea urchin, isolated soy proteins, papaya, and N-acetyl-glucosamine. The amount of the probiotic glycoproteins of the total weight of a composition on dry basis is preferably more than 10 weight percent.

Preferred amounts of N-acetyl-glucosamine as weight percent shall be in the range of from about 10 to 30 percent, for example such as 20 weight percent.

Accordingly, weight ratio of isolated soy proteins is preferably more 1.0, for example 1.15.

Water processed soy proteins retaining a natural level of isoflavones are preferable for mixing with probiotic glycoprotein. Yet, most preferable is sea urchin protein predigested with papaya latex and mixed with papaya. Freeze drying is considered as most appropriated procedure for preparation of powdered form of each ingredient.

Alternatively, the present invention provides a drink where probiotic glycopeptides are added to dealcoholized red wine. Reverse osmosis can be implied to remove alcohol from red wine probiotic and soy aminoacids serve as a daily source of protein.

The proposed composition can be served as a powder mixed with milk, orange juice or other beverage of choice.

### DESCRIPTION OF THE PREFERRED EMBODIMENTS

Specific production embodiments are presented hereinafter.

### EXAMPLE 1

Isolation of Glucopeptides from Lactobacillus Plantarum

1. Biomass Preparation

b 15 kg of wet biomass was supplied by Chr. Hansen Corp., (Milwaukee, Wis.) Biomass was separated from the rest of the feeding media by washing with distilled water three times. Wet biomass was rinsed twice by 15 L of distilled water using centrifuge Backman JM-6 at 3900 rpm/min until supernatant liquid becomes colorless.

2. Hydrolysis.

3 kg moist biomass was resuspended in 20 L H₂O and boiled for 15 min After that, it was diluted in 30 L H₂O+NaHCO₃ (to achieve pH=6. 0) and added 30 g of lyzosyme (Canadian Inovatech, Inc., Vancouver, Canada). Hydrolysis was done for 48 hours at 54° C. Then, 500 ml food grade vinegar was added to achieve pH=4.0 and was centrifuged on Beckman JM-6 g at 4000 rpm for 5 hours.

3. First Ultrafiltration.

Cartridge with the membrane capable of retaining compounds with molecular weight less than 3000 D and with S=0.09M² at speed 2.5 L/h (Millipore Corp, USA) were used. 8.8 L solution with retained nuclear acids, phospholipids, and lysozyine was wasted.

4. Second Nanofiltration.

Cartridge with Nanomax® membrane installed on Prolab System, (Millipore Corp., USA) was used for filtrating compounds with molecular weight less than 300 D (muramylpeptides, salts, acetic and lactic acids). Then, 5 L of retained was freeze dried. Yield was 110 g of molecular peptodoglycans with the molecular weight in the range of 300-3000 D.

### EXAMPLE 3

LDH Assay of TNF Alpha Cytotoxicity.

Lactate dehydrogenase (LDH) is a stable cytosolic enzyme, product of a housekeeping gene that is released upon cell lysis. Released product is measured in rapid enzymatic assay, measuring the conversion of tetrazolium salt into a red formazan product. The reaction have two steps: LDH is producing NADH which is reducing in the next step the salt to a red product. LDH assay has been used for variety of applications with many different cell types for measurement of cell mediated cytotoxicity, mediated by chemicals or other agent as well as changes in the total cell number.

In our experiments we used LDH release assay (CytoTox 96. Promega) in order to asses the TNF induced cell death and consecutively the cytoprotection provided by certain compound to the TNF and FAS induced cytolysis. The supplier of the kit recommended the procedure we used.

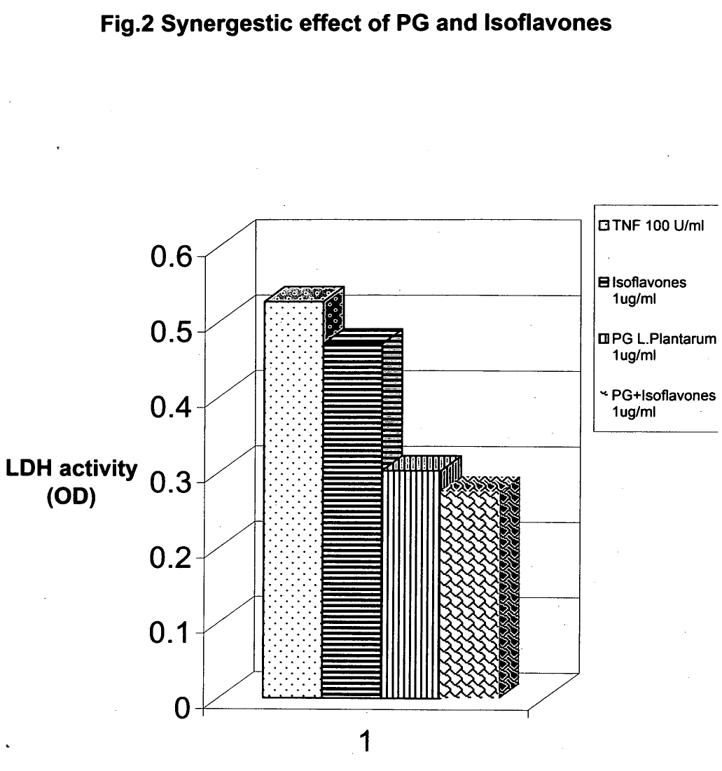

Briefly: A549 cells (human lung cancer) were seeded in six-well plates, and after 24 h (70% confluence) treated with 25 ug/ml cycloheximide (CHX) and either 100 U/ml human TNF (Beoringer) or. Twenty hours after the treatment 20 ul of the cultured supernatant was removed and tested for the LDH activity in 96 well plates in triplicate. Samples were assayed on an EL340 Microplate reader (Biotec Instruments, Inc) at 490-nm wavelength. FIG. 1 represents the results of inhibition of LDH release by 1 μg/ml of peptidoglycan derived from L. Plantarum.

FIG. 2 demonstrates the synergistic effect of soy isoflavones and peptidoglycans on LDH inhibition.

### EXAMPLE 3

Effect Disaccharide Dipeptide on NF-κB Expression.

NF-κB p50 ELISA assay was done on A549 human lung carcinoma cells. The samples were pools of duplicate nuclear extracts prepared from duplicate plates of treated cells and stored at −80° C. The same amount of protein was added to the plate for each sample, as determined by Bradford protein assay (mean of two assays). All sample dilutions, control, and blank were tested in triplicate wells. The results given are the mean values. The primary antibody dilution was 1/2000 and the conjugate dilution was 1/50 K. The values were read on the luminometer with a 1 second integrated reading.

TABLE 1
Results of p50 ELISA

| | Raw RLU, | Blanked RLUs | |
|---|---|---|---|
| | 250 ng | 250 ng | Fold increase |
| Treatment | Protein/well | prot/well | over treatment |
| No treatment | 17,453 | Blank | N/A |
| TNF alpha/CHX | 224,696 | 207,243 | 12.87 |
| GMTP, 2.5 μ/ml | 16,227 | −1,226 | 0.93 |
| GMTP, 10 μ/ml | 22,474 | 5,021 | 1.29 |
| GMDP, 2.5 μ/ml | 11,470 | −5,983 | 0.66 |
| GMDP, 10 μ/ml | 12,748 | −4,705 | 0.73 |

Assay information:
Average RLU for background wells (complete lysis buffer) = 4447
Average RLU for positive control wells (Jurkat nuclear extract) minus background = 209,546
CHX—cycloheximide
RLU—relative light unit

### EXAMPLE 4

Effect of Biodegradable Probiotic Glucosaminemuramyl Peptides on Hepatitis C and Hepatocellular Carcinoma.

Case 1.57 year old Caucasian female with hepatitis C, ammonia intoxication, and diabetes did diteriate on Interferon alpha treatment. Her viral load was increased from g1.4M to 4.2M copies and quality of life was deteriated significantly after hospital stay. She was placed on 10 mg probiotic glycopeptides daily. Her blood glucose level was lowered immediately, fatigue was reduced, as well as viral load to 900K copies within next two month.

Case 2.71 year old Caucasian male with hepatitis C hepatocellular carcinoma with lung metastasis was placed on 150 mg probiotic glycopeptides daily. His GGTP level was reduced two fold from 147 U/l to 74 U/l, whit blood cell and platelets count was improved by 70% and 44% within first 24 hours. His viral load was reduced from 10.7M to 1.1M after 6 month of administration of 50 mg/daily of this glycopeptides.

### EXAMPLE 5

Effect of Probiotic Glycopeptides on Ulcerates Colitis and Leukemia.

Case 1.57 years old Caucasian female with ulcerates colitis was placed on 10 mg of glycopeptides daily. Her gastrointestinal discomfort was improved and she had a reduced need for conventional drug therapy, i.e. steroids and asulfodine.

Case 2. A Caucasian male with chronic lymphocyte leukemia was placed on 20 mg of glycopeptides daily. His white blood cell count was reduced from 123000 to 910000 within first two weeks of feeding

### EXAMPLE 7

Dietary Management of Aplastic Anemia.

The patient S. 18 years old, was complaining on significant fatigue and prolonged bleeding during her menses. Objectively, she has had hemorrhagic petechiae in the skin all over her body. Aplastic anemia was diagnosed based on the results of bone marrow biopsy. Epstein-Barr virus was detected and believed to be a cause of bone marrow anaplasia. She was placed on high doses of prednizone 350 mg daily, neoral 200 mg daily, cyclosporine 200 mg daily, Neupogen ®, and erethpoietin. Her condition was steadily deteriorating regardless of this treatment. One year later she started taking peptidoglycan of L. Plantarum, prepared accordingly to the example. The average dose was 1 g per day. The blood CBC results are presented in the table 8.

TABLE 8
Effect of probiotic peptidoglycans on blood
CBC in the patients with aplastic anemia.

| | Platelets, | WBC, | RBC | | | | Neutrophils, | |
|---|---|---|---|---|---|---|---|---|
| Date | Thou/cm | thou/cm | million/cm | Hb, g/dl | Hematocrit | | Abs. Aut. | |
| Feb. 15, 2001 | 22000 (L) | 2100 (L) | 2.56 (L) | 7.9 (L) | 24.5 | (L) | 1.1 | (L) |
| Feb. 19, 2001 | 11800 (L) | 4100 (L) | 2.36 (L) | 7.79 (L) | 23.1 | (L) | 2.7 | |
| Feb. 22, 2001* | 20000 | 2300 (L) | 2.43 (L) | 8.2 (L) | 24.1 | (L) | 1.2 | (L) |
| Feb. 26, 2001 | 17800 | 4000 (L) | 2.25 (L) | 7.69 (L) | 22.5 | (L) | 2.6 | |
| Mar. 5, 2001 | 18700 | 2300 (L) | 2.33 (L) | 8.10 (L) | 23.8 | (L) | 1.4 | |
| Mar. 12, 2001 | 29200 | 2100 (L) | 2.61 (L) | 8.9 (L) | 27.4 | (L) | 4.7 | |
| Mar. 19, 2001 | 19900 | 3700 (L) | 2.39 (L) | 8.48 (L) | 25.3 | (L) | 2.5 | |
| Mar. 26, 2001 | 25000 | 3900 (L) | 2.40 (L) | 8.2 (L) | 25.4 | (L) | 2.6 | |
| M | 20489 | 3100 (L) | 2.13 (L) | 8.12 (L) | 24.3 | (L) | 2.1 | |
| Apr. 2, 2001 | 23000 | 4000 (L) | 2.46 (L) | 8.85 (L) | 26.5 | (L) | 2.7 | |
| Apr. 9, 2001 | 23000 | 4200 | 2.46 (L) | 8.92 (L) | 26.5 | (L) | 2.8 | |
| Apr. 16, 2001 | 22000 | 2000 (L) | 2.41 (L) | 8.75 (L) | 26.0 | (L) | 0.9 | |
| Apr. 23, 2001 | 28000 | 4700 | 2.66 (L) | 9.59 (L) | 29.0 | (L) | 3.2 | |
| Apr. 30, 2001 | 38000 | 5100 | 2.99 (L) | 10.2 (L) | 31.4 | (L) | 3.7 | |
| M | 26800 | 4000 (L) | 2.58 (L) | 9.27 (L) | 27.98 | | 2.66 | |
| May 09, 2001 | 33000 | 2800 (L) | 2.78 (L) | 10.2 (L) | 30.0 | | 49.2 | |
| May 21, 2001 | 42000 | 2400 (L) | 3.09 (L) | 10.8 (L) | 32.8 | | 46 | |
| Jun. 4, 2001 | 39000 | 2500 (L) | 3.06 (L) | 10.6 (L) | 32.0 | | 48 | |
| Jun. 18, 2001# | 50000 | 2200 (L) | 3.01 (L) | 10.5 (L) | 31.4 | | 48 | |
| M | 41000 | 2470 | 2.98 | 10.5 | 31.5 | | 43.82 | |
| Sep. 4, 2001& | 47000 | 3100 (L) | 2.89 (L) | 10.2 (L) | 30.5 | (L) | 1.7 | (L) |
| Oct. 2, 2001 | 60000 | 3700 (L) | 3.17 (L) | 11.6 (L) | 33.4 | (L) | 1.9 | |
| Oct. 30, 2001 | 61000 | 3500 (L) | 3.25 (L) | 11.4 (L) | 34.2 | (L) | 2.0 | |
| Dec. 3, 2001 | 68000 | 5200 | 3.36 (L) | 11.6 (L) | 35.3 | (L) | 3.0 | |
| Jan. 14, 2002 | 67000 | 4000 (L) | 3.48 (L) | 12 | 36.1 | (L) | 2.0 | |
| Feb. 25, 2002 | 75000 | 4700 | 3.65 (L) | 12.5 | 38.4 | | 2.9 | |

*first day on peptidoglycans of L. Plantarum, average dose of 1 g per day
#cyclosporin was reduced to 50 mg/day with concurrent reduction of steroids.
&She was off cyclosporine and steroids

One can see the steady rise in the count of platelets, WBC, hemoglobin, neutrophils, and hematocrit after starting peptidoglycans of L. Plantarum.

We claim,:

1. A method of reduction of oxidative stress under all conditions with elevated gamma glutamyl transferase and NF-κB in humans and domestically useful animals with concurrent enhancement of apoptosis of cancer cells and cells infected with RNA virus, comprising the administration of therapeutically effective amount 10-5000 mg glucosaminemuramyl peptides.

2. A method of dietary management of disease state with elevated gamma glutamyl transferase by feeding humans and domestically useful animals with predigested antioxidant food, comprising administration a therapeutically effective amount of N-acetyl-glucosaminemuramyl peptides, bioflavonoids, papaya, and sea urchin.

3. A method of claim 3 where a disease state is caused by liver, brain and lung metastasis metastasis of colorectal, ovarian and breast carcinoma

4. A method of dietary management of anemia, thrombocytopenia, and leucopenia by feeding humans and domestically useful animals with therapeutically effective amount of N-acetyl-glucosamine-N-acetyl-muramyl-L-alanine,D-isoglutamine-L-lysine-D-alanine.

5. A method of claim 3 where the disease state is glioblastoma multiforme.

6. A method of claim 3 where the disease state is hepatitis C.

7. A method of claim 1 where disease state is osteoporosis.

8. A method of claim 3 where disease state is caused by anesthetics.

9. A method of claim 3 where the disease state is caused by opiates.

10. A method of claim 3 where a disease states is caused by alcohol.

11. A composition of claim 1 where disease state is hypertension.

12. A composition of claim 1 where disease state is bone metastasis.

13. A method of claim 3 where a disease state is myielodisplastic syndrome.

14. A composition of claim 1 where a disease state is Parkinson disease.

15. A method of claim 3 where the disease state is melanoma.

16. A method of claim 1 where probiotic glucosaminemuramyl peptides are, administered in combination with selenium, vitamin C, vitamin B12, vitamin B6, vitamin D, calcitriol, alphacalcidol, folate, androgen, carnitine, and beta carotine.

17. A composition of claim 1 where a disease state is hepatocellular carcinoma.

18. A method of claim 4 where a disease state is rheumatoid arthritis and autoimmune hepatitis.

19. A method of claim 4 where a disease state is diabetes type 1 and 2.

20. A method of claim 1 where a disease state is leukomia
